(19) United States
(12) Patent Application Publication
(10) Pub. No.: US 2024/0382960 A1
(43) Pub. Date: Nov. 21, 2024

(54) MASSIVELY PARALLEL SINGLE-CELL SEQUENCING WITH MAPPED CELLULAR OBSERVATIONS

(71) Applicant: Thaumachron LLC, Rapid City, SD (US)

(72) Inventor: ANDREW SIMPSON, Rapid City, SD (US)

(21) Appl. No.: 18/668,650

(22) Filed: May 20, 2024

Related U.S. Application Data
(60) Provisional application No. 63/467,523, filed on May 18, 2023.

(51) Int. Cl. B01L 3/00; C12N 15/10; C12Q 1/6874; C12Q 1/6876

(52) U.S. Cl. CPC B01L 3/502761; C12N 15/1096; C12Q 1/6874; C12Q 1/6876; B01L 2300/0663; B01L 2400/0487; B01L 2400/0605

(57) ABSTRACT

This invention pertains to a novel method of single cell sequencing where cellular observations are paired directly with sequences. This method consists of a sequencing element with barcoded solid-phase PCR probes, cellular wells, and an optional cellular trap. Cells are loaded with reagents directly into wells or captured with cellular traps where the cellular wells are then lowered over the trapped cells. The sequencing element is then added. Cellular observations such as binding and affinity are then recorded. Cells are lysed and RT-PCR is performed to create a cDNA library that is then sequenced. Barcodes in the library allow sequences to be mapped back to individual wells. This method has applications from basic biology to personalized drug discovery to creating large databases for machine learning training sets.

### CROSS-REFERENCE TO RELATED APPLICATIONS

This application claims priority pursuant to 35 U.S.C. 119 (e) to U.S. Provisional Patent Application Ser. No. 63/467,523, filed May 18, 2023, the entire disclosure of which is incorporated herein by reference.

### FIELD OF THE INVENTION

The present invention relates generally to cell sequencing. More specifically, the present invention is concerned with methods and corresponding devices for single-cell sequencing, and the beneficial use of single-cell sequencing, the collection of cellular observations mapped directly to individual cells.

### BACKGROUND

The human genome project was launched in 1990 and completed in 2003 requiring enormous effort and resources. Early sequencing technologies such as Sanger sequencing were used for the human genome project. This sequencing method, although highly accurate, is slow and has limited throughput. This sequencing method still has certain use cases, but many modern tasks utilize next generation sequencing methods that allow for the significant benefit of massively parallel sequencing where many sequences can be processed in parallel in a high throughput manner. Since the completion of the human genome project, the time and cost to sequence a genome have dramatically decreased. Illumuina sequencing works through a sequencing by a synthesis approach using bridge amplification and fluorescently labeled nucleotides that can be captured by an imaging sensor with high accuracy. A key drawback of this method is that only fairly short sequences can be read; however, new methods allow for sequencing of much longer sequences. These new long read methods, such as PacBio and Oxford Nanopore, can sequence full length transcripts, but come with the drawback of lower accuracy although the latest methods of long read sequencing have greatly improved accuracy.

Traditional methods of sequencing rely on pooling multiple cells and extracting DNA or RNA. The traditional bulk sequencing is useful for many applications; however, many cell populations are heterogeneous and benefit from sequencing individual cells. Single-cell sequencing is a critical tool for many areas including cancer research, cell population variants, and detecting rare cell types. Multiple methods exist such as Smart-seq, Drop-seq, Chromium 10×, and Seq-Well. Currently, droplet based methods, such as Chromium 10×, are the most effective for most use cases. Chromium 10× sequencing uses oligonucleotide coated beads. These oligonucleotides contain molecular barcodes that are unique to each bead, Unique Molecular Identifiers (UMIs), a shared primer site, and an adapter site. UMIs are random oligonucleotides added and attached to beads, and they are used to control for PCR bias and de-duplication of sequencing data.

Recently, sub-nanoliter cellular wells have become available. Cells are added on top of these wells and through methods such as rocking the cells will cause cells to fall into wells. If the well size is only slightly larger than the cell size, then each well will only contain one cell. These types of cellular wells have uses in isolation of single cells, growing colonies from single cells, and capturing interactions between multiple cells.

Seq-Well is a form of single cell sequencing that uses barcode labeled beads similar to Chromium 10×. Seq-well uses sub-nanoliter cellular wells to capture cells and is loaded with one bead per well. Cells are loaded into the cellular array with the beads and a library preparation method similar to Chromium 10× is used. This method has similar advantages to the Chromium 10× droplet based method, and in some cases it is more cost effective; however, this method does not know which bead is in which well making direct mapping of cellular observations not feasible.

DNA microarrays contain many oligonucleotide probes usually on a glass substrate. These are created using lithographic methods or direct printing using technology similar to ink-jet printers. These microarrays can be used to detect gene expression in cells, SNPs, fusion genes, and more. An extension of microarray technology is solid phase PCR. This technology consists of a molecular spacer, such as hexaethyleneglycol (HEG), to avoid steric hindrance of DNA polymerases from running into the substrate, followed by a primer sequence, and a complementary sequence to target(s) of interest. In the case of mRNA sequences, reverse transcriptase is used to extend the probe to the complementary sequence of the mRNA. The extended probe then either has another adapter sequence or a PCR primer on the other end. After this step, PCR can be used to exponentially expand the target(s) of interest.

In many cases, individual cells have properties that can be observed with different methods either directly or indirectly. For example, T-cells, B-cells, or recombinant yeast libraries expressing proteins that are unique to each cell (TCRs, antibodies, nanobodies, etc.) can be monitored to see if they bind to target(s) of interests through fluorescence or methods. Affinity can be measured by varying the concentration of the targets of interests. Fluorescence-activated cell sorting (FACS) can be used to separate out cells into populations of interest. Individual cells can then be picked and cloned for sequencing. Additionally, different concentrations of various compounds are important for cell differentiation and other functions. For example, larger cellular wells containing multiple cells can each be given a different concentration of a substance and then sequenced to measure differential gene expression. In any case, any current method of mapping cellular observations has a very limited scope of mapping genomic diversity to cellular observation as only a tiny minority of cells can be sequenced. What is needed is a high throughput single-cell sequencing method that allows for mapping of cellular observations. Against this backdrop embodiments in accordance with this invention were developed.

### SUMMARY

The objective of this invention is to solve the need for a massively parallel single-cell sequencing method that has mapped observations for individual cells. This will allow for the inexpensive construction of large scale cellular databases with observations mapped to genotypes or transcriptomes. This has applications from basic biology, to personalized drug discovery, to machine learning datasets for drug creation.

By combining an aligned sequencing element array, cellular well array, and a method of observing cellular properties, such as, but not limited to, optical observation, fluorescence, or interaction with magnetic fields, novel methods of single cell sequencing with mapped cellular observations are provided.

The sequencing element array includes solid phase PCR probes that are added onto a substrate that align with cellular wells. The probes comprise a spacer, to avoid steric hindrance of the polymerase used in the reaction, a shared PCR primer, a molecular barcode, an optional UMI, and an adapter sequence. Either the 3′ or the 5′ end of the probe is affixed to the spacer. The sequencing element may contain a small hole or holes and one side may be covered with a semipermeable membrane.

Cells are then added to the wells following best practices along with reagents for the solid phase PCR and reverse transcription reactions. In some embodiments, cells are first captured on cellular traps such as, but not limited to, modified protein microarrays that have features sizes where only single cells can bind that are aligned to the design of the cellular well array. The wells are then aligned and connected for a tight seal.

Cellular observations are performed on the loaded device. Lysis of cells is then performed using methods such as thermal lysis or chemical agent lysis through the semipermeable membrane or in some embodiments microfluidic valves. After cellular lysis, the reverse transcriptase reaction occurs and best practice thermocycling for the PCR reaction is performed. The assembly is then disassembled and the resulting cDNA product is collected for sequencing. Following best practices, this cDNA library is then sequenced. The molecular barcodes are used to determine which sequences belong to each well. In this way, cellular observations are mapped to sequences in a massively parallel way.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION

Embodiments of the present invention are described in detail below with reference to the attached drawing figures, wherein:

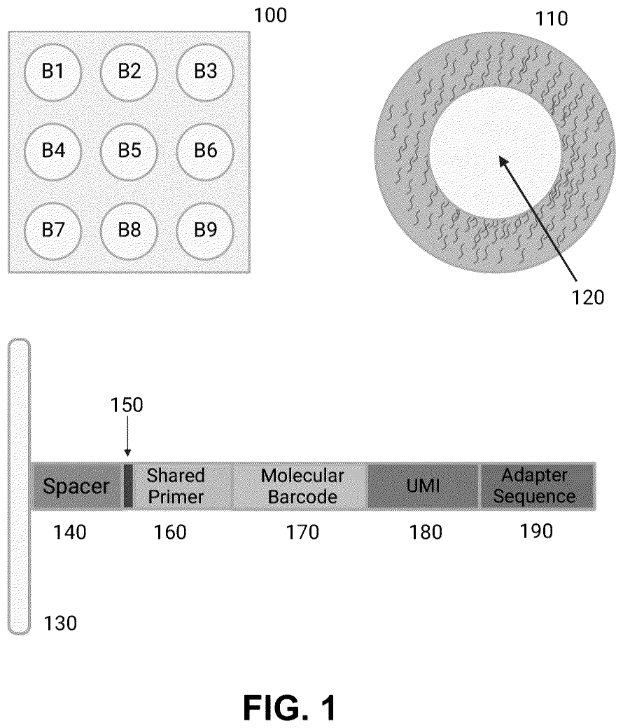

FIG. 1 Solid Phase PCR probes with molecular barcodes, general design;

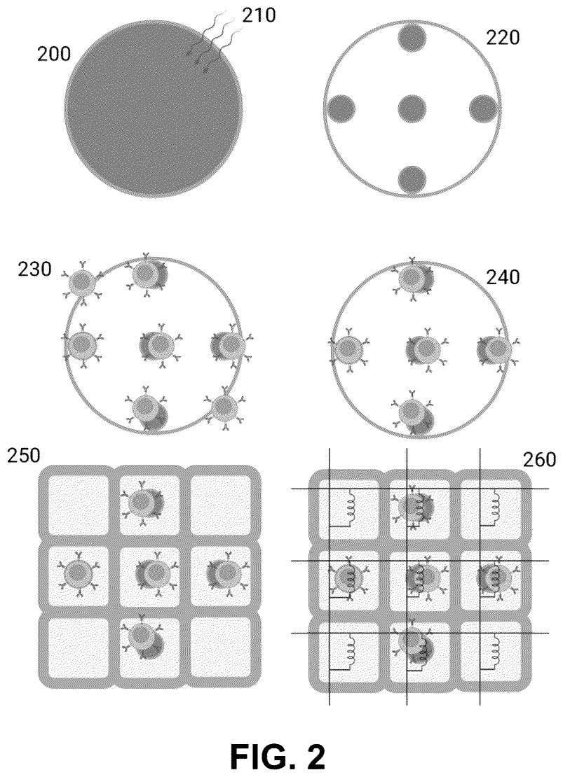

FIG. 2 Cellular traps by laser ablation and magnetic affinity observations by individually addressable electromagnets.

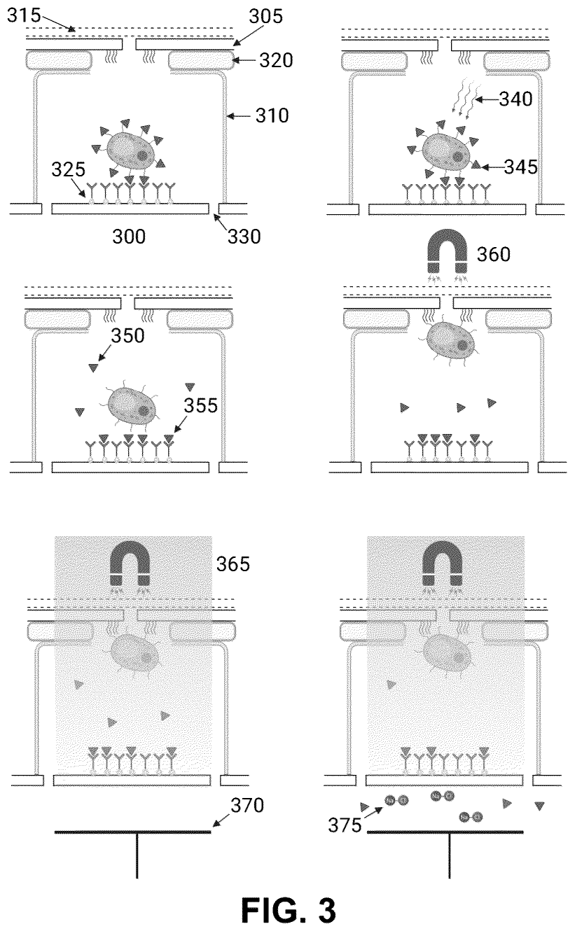

FIG. 3 Protein-Protein affinity measurements with photolytic cleavage and localized surface plasmon resonance.

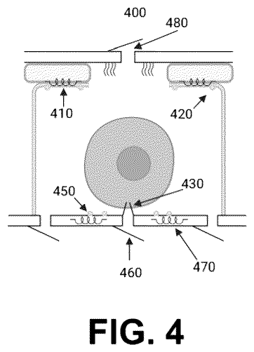

FIG. 4 Potential configuration options for cellular wells.

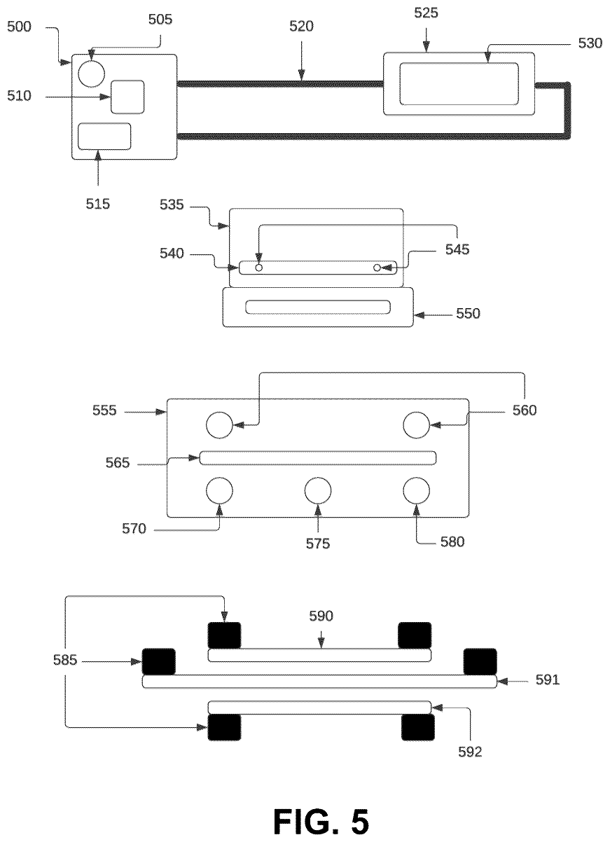

FIG. 5 Environmental control device for laser ablation, thermal cycling, and incubation.

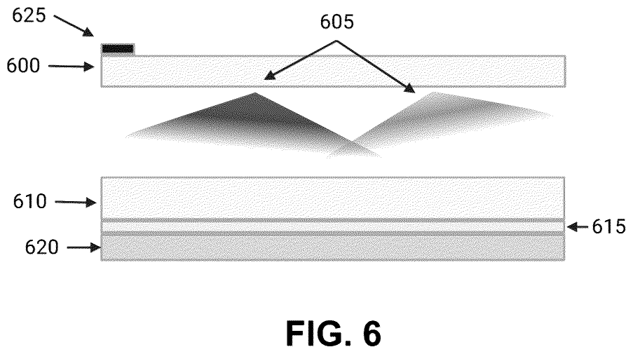

FIG. 6 Potential lenless microscopy options.

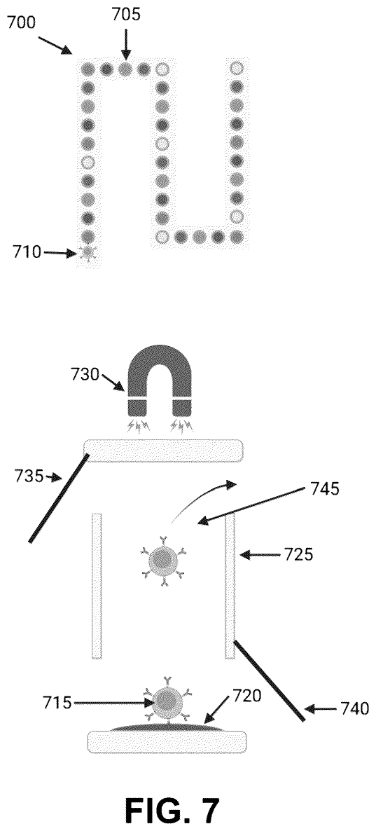

FIG. 7 Linear protein trap.

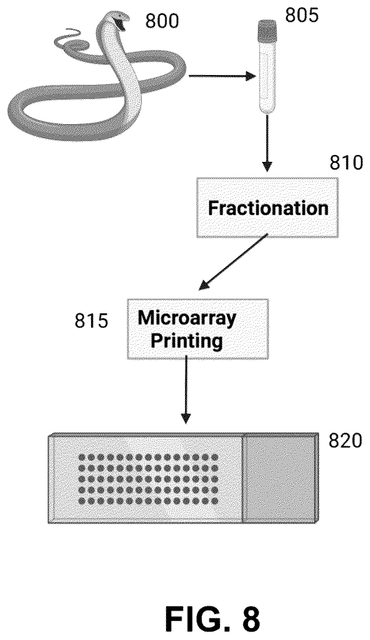

FIG. 8 Venom protein and peptide microarray.

The drawing figures do not limit the present invention to the specific embodiments disclosed and described herein. The drawings are not necessarily to scale, emphasis instead being placed upon illustrating the principles of the invention.

### DETAILED DESCRIPTION

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

This disclosure relates to novel methods of massively parallel single cell sequencing. Unlike other methods, these methods allow for cellular observations, any measurement of a cell or cells, to be mapped to individual cell sequences. A key use case of this methodology is creating high throughput databases of high diversity proteins, such as antibodies and T-cell receptors. Using cellular traps, described later, single cells with high diversity receptors can bind to targets of interest. Trap cells are observed and sequenced using the methods to be described. Using cellular traps in combination with the sequencing methods herein allows for the collection of data including whether a receptor binds to a target, affinity of the receptor and target, and sequence of the receptor. This method is also useful for the study of cellular transciptomics under different conditions. The system is compatible with a myriad of different microfluidic options depending on applications such as, but not limited to, valves, pumps, electrodes, and electromagnets.

Core embodiments described herein comprise sequencing elements containing affixed barcoded probes, a cellular well array, and in some embodiments a protein, or other target of interest, and a microarray. In one embodiment cells of interest are loaded into cellular wells along with reagents required for sequencing, such as, but not limited to, DNA polymerase, reverse transcriptase, and DNA primers. Observations of the cells, such as fluorescence at different wave lengths, are recorded before or after the sequencing element added and aligned precisely to assure that no mRNA will transfer between wells enabling single-cell sequencing to ensure the barcode matches sequences of cells in only one well.

Cellular observations are performed using optical microscopy including fluorescence in most cases. In some applications other methods such as ultrasonic imaging, electrodes (created for each well or regions of wells), may be used. Imaging of the wells is performed using a microscopy where cellular observations are made of all cells in the field of the microscope and stitched together using image stitching best practices. Additionally, other methods such as scanning or wide area high lensless microscopy (allowing for all wells to be observed at once) may be used.

The sequencing element shown in FIG. 1 is constructed using an appropriate substrate 130 (generally a few types of glass, but can be any material compatible with an aqueous environments and temperatures up to approximately 100° C.) with an appropriate molecular spacer affixed 140, such as hexaethyleneglycol or nucleotides, that allow enough spacing to avoid steric hindrance of DNA polymerases from running into the substrate. In some embodiments the sequencing element is created by adding probes directly to the bottom of the cellular wells. Probes are then built using best practices, such as lithography or printing, and are placed with the same spacing as the sub-nanoliter cellular wells used in the next step with sizes smaller than each well 100. Either the 3′ or the 5′ end of the probe are affixed to the spacer 140. This spacer 140 is designed to have enough distance to mitigate steric hindrance of polymerases. In some aspects, the next feature of the probe is an optional endonuclease cleavage site 150. In addition, the next feature is a common primer sequence 160 that is shared by all probes. This shared primer is followed by a molecular barcode 170 that is unique to each probe. Each barcode has a known spatial location on the sequencing element and barcodes optionally are designed using Hamming codes or other methods of error correction. For example, in FIG. 1 100 the barcode B1 is different from B2, and so forth. The next part of the probe is an optional UMI 180. These UMIs are a collection of randomly mixed oligonucleotides that are added to the entire array. Each probe will contain a mix of multiple UMIs. These UMIs are used for detection of PCR amplification bias. The last feature of the probe design is an adapter sequence 190. Depending on use, the adapter sequence may be, but is not limited to, a specific to a target of interest, a common overhang for tailing of reverse transcriptase, or primer for a ligase reaction to bind the probe to the sequence(s) of interest. In some embodiments, the sequencing element also contains a micron scale gasket that seals off each well or may be fused or glued to cellular wells.

In some cases, such as photolithographic methods of creating DNA microarrays, the length of probe may be limited. Sequencing arrays can be created with components up to and including the molecular barcode. Between the molecular barcode 170 and UMI 180 a common sequence can be added. DNA strands produced in other methods containing another common sequence, UMI (optional), and adapter sequence can be attached to the affixed sequence containing the barcode using a ligase bridge for example. Additionally, multiple different adapter sequences can be used allowing for a much larger number of specific targets to be sequenced at once.

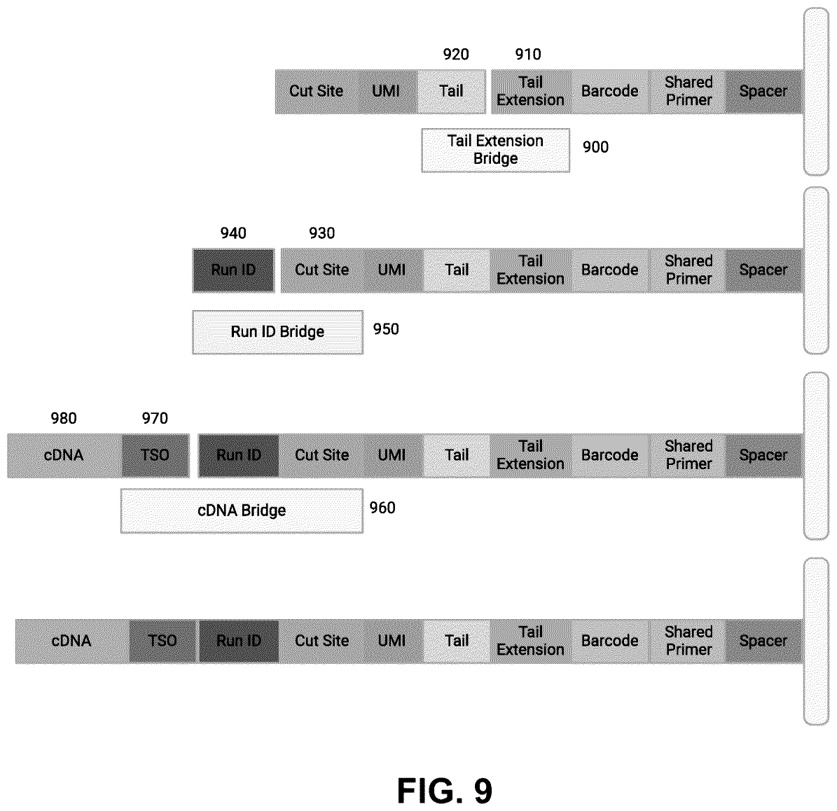

In some embodiments sequencing arrays can be made reusable through use of a run ID as shown in FIG. 9. Additionally, UMIs made from random nucleotides, or pseudo-random nucleotides lacking complementary regions for primers, cut sites, etc., can be constructed by a PCR with DNA ligase reaction with two sets of complementary DNA sequences with known over hangs that can be ligated to the random nucleotides. PCR can then be used with the ends of the known sequences. These new DNA segments with random sequences in the middle can be used for UMIs. A ligase bridge 900 is complementary to a tail extension sequence on the affixed probe 910 and a tail sequence of the UMI 920. Ligase can be used to covalently bond the two segments together. Importantly, the other known sequence of the UMI sequence is a site specific cut site 930 allowing for CRISPR or other site specific endonucleases to cut the probe sequence and allow it be reused. Using another ligase bridge 950, the probe is covalently attached to a run ID segment 940, a sequence unique for each time the array is used. This run ID segment 940 allows for reusability. For example, using the cut site 930 without the run ID runs the risk of incomplete cuts of every probe. This lack of complete cuts could cause issues in downstream analysis. With the run ID sequence 940, any run ID sequences that do not match the current run can be excluded. Additionally, in embodiments, these run IDs can contain similar error correcting mechanisms as further described herein. In cases with the 3′ end of the probe affixed to the substrate, another ligase bridge 960 can be used to capture cDNA 980. This bridge is complementary to the run ID 940 and potentially all or some of the cut site 930 and the complement (including extra nucleotides from terminal deoxynucleotidyl transferase (TdT) if necessary) of the TSO (template switch oligonucleotide) (used in many cases to create a the first cDNA strand from mRNA in combination with site specific primers or a poly T primer for any mRNA). This approach allows for one or more expressed sequences of interest or whole transcriptomes to be collected.

Additionally, multiple probes can also be constructed for individual wells. Using laser drilling or lithographic methods, small holes are created, as shown in FIG. 1 120. With the gasket, fluid containing reagents will flow to each subsequent layer. In some aspects, a semipermeable membrane is added where small reagents such as, but not limited to, nucleosides, salts, etc. are added. In another aspect, microfluidic or nanofluidic methods are used to individually address each well such as an electronically controllable check valve or pump and may be used in concurrence or without the semipermeable membrane. Depending on technology of probe construction, each layer of the sequencing element, including only a single sequencing element, could have multiple species of barcoded probes per well.

Sub-nanoliter cellular wells are designed using best practices such as lithography, laser drilling, or laser induced deep etching. In some embodiments, wells are first loaded with cells and in others cellular traps or other methods are used to capture and select cells. In the case of cells captured through another method, the wells are designed to have holes through the substrate. In one aspect, the sequencing element is placed over and aligned to the wells. In another aspect, the through hole wells are aligned and combined with the sequencing element and aligned and placed over the cellular traps. Methods of alignment depend on manufacturing tolerances and may include, but are not limited to a flexture mechanism, precisely aligned grooves, through holes with pegs, hydrophobic dots or other patterns, magnetic seals, or electromagnetic seals (allowing levitation between parts).

Cellular traps shown in FIG. 2 are created using laser ablation 210 of larger features 200 such as those on protein microarrays to create multiple smaller features that only one cell can bind to 220. After creating cellular traps, cells of interest, such as yeast display cells or B-cells, are passed over the traps following best practices such as rocking 230. Non-binding cells can then be washed away leaving only the cells left in traps 240. Cellular wells can then be placed over cellular traps 250. Optionally, individually addressable electromagnets 260, either per cell or per region, are used for manipulating cells. Laser ablation may lead to rough surfaces. In some embodiments, laser polishing or other polishing methods are used to obtain smooth flat surfaces to avoid leakage of RNA and DNA between wells. In other embodiments, laser tomography of other parts that may not be suitable for laser polishing, is used which allows for cellular wells to exactly match other parts.

Protein and other microarrays are often stored at ultra low temperatures, −86° C. to −45° C., and with very low humidity. Laser ablation or other methods are usually done at room temperature with higher than required humidity. A small form factor cooling device, for example as shown in FIG. 5, using thermal electric cooling (TEC) 510 to cool the protein microarray during creation of single-cell traps is used. In some aspects, a water block on the hot side of the TEC is connected to a pump and radiator or a heat pipe air cooler radiator 500. The cold side is connected to a water block 510. The water block is connected to a pump and reservoir 505 of a potassium acetate solution or other appropriate coolant. In some embodiments, the water blocks are 3D printed with gyroid structures to maximize heat transfer while minimizing space. Temperature appropriate insulated tubing 520 is used to connect to a 3D printed or otherwise machined part 525 designed to fit the microarray 530 in a thin form factor. Additionally, a solid state or other low vibration fan 550 is combined with a desiccant, such as silica gel or molecular sieves 535, a separate water block 545 and radiator 540 used to produce dry cold air. A desiccant wheel may be used in cases where dry air is needed for longer periods of time. This dry cold air flows over the microarray during ablation. Nitrogen or other dry gases may also be cooled and used. Thermocouples and standard microcontroller components are used to maintain proper temperatures and humidity. The microcontroller is controlled or programmed with USB, WiFi, or other methods. In some embodiments, the device is powered by a battery 515. The system is pre-chilled before the microarray is added for ablation.

Embodiments can also include, individually addressable micro-electromagnets made in combination with cellular wells, either on the same substrate or another aligned transparent substrate. Using photolithography methods, individually addressable electromagnets are created for each well using a coil, rosette, or other shape that allows a through hole. In some embodiments, microfluidic channels are added near the electromagnets. These channels are used for cryogenic coolants allowing for superconducting electromagnets. Additional channels are used to assure the correct temperature of cellular wells is maintained.

In some aspects, affinity information is calculated for cells with magnetic properties. Cells are loaded with magnetic substances or engineered to be magnetic. With a differential in a magnetic field, either from the optional substrate electromagnets 260 or externally through another device such as an electromagnet on the end of a microscope objective, cells are removed from their cellular traps. Knowing the strength of the magnetic field, as well as how magnetic a cell is and density of receptors and target of interest, the affinity of receptor and target is calculated by deformation of the cell or how much magnetic field strength is needed to remove the cell from the trap. These calculations are done in multiple ways such as magnetic simulation software and mathematical approaches. In some aspects, magnetic simulations are used as a training set for artificial intelligence (AI) models to predict affinity. In some aspects, AI models and other methods are experimentally verified using micropipette adhesion affinity measurements on trapped cells. Another option for a training set for AI models would be collecting micropipette adhesion affinity measurements under different magnetic field strengths.

Cellular traps can also be used to select clones by washing away cells that are not trapped and allowing trapped cells to clonally expand. The cloned cells can then be washed away and expanded. Using cellular traps with multiple traps of the same protein and using variable strength electromagnetic confinement where cells with weaker affinity are pulled off are used as a form of affinity maturation. This method leaves only strong binders, i.e., relatively high affinity for each other, behind. In some aspects, the process is repeated to select high affinity clones. If the process is done with protein microarrays that contain most of a proteome, cross-reactivity is minimized as well through this process by removing cells that bind to elements of the proteome and keeping those that flow through.

The use of cellular traps combined with cellular wells and the sequencing element array allows for collection of binding and affinity data for potentially hundreds of thousands of novel cells, or more depending on the size of components.

Direct measurement of binding kinetics and affinity of receptors and targets, see FIG. 3 is done with surface plasmon resonance (SPR) or localized surface plasmon resonance (LSPR) and photolytic cleavage of receptors (in the case of SPR, an additional prism feature is needed). As before, a sequencing element 305 with an optional semipermeable membrane 315 and micron scale gasket 320 are combined with cellular wells 310. Protein, or other targets of interest, traps are constructed in a similar way as before, but using either a gold or other suitable metal coated substrate or target covered gold nanoparticles are affixed to a substrate 325. Single cells will bind to cellular traps and wells aligned as previously mentioned. The receptor of interest in these cells contains a photolytic cleavage site 345 that is then exposed to the correct wavelength of light to trigger cleavage 340. Magnetic fields, either externally or through localized micro-electromagnets, are used to pull the cleaved cell to the top of the well 360. If the receptor is also fluorescent or a fluorescent antibody is included that binds to part of the receptor, the concentration of the receptor is determined 350 355. Laser light is shown through the device assembly at the correct wavelength or angle where a sensor 370 records changes in angle or wavelength. Through diffusion, pumping, or other methods 375 the concentration of the receptor will decrease over time and be removed through a microfludic channel. Monitoring of the change in angle or wavelength properties in each well such as Ka, Kd, and affinity are recovered for the receptor and the target.

In some aspects, standard microscopy techniques are used to record observations; however, lensless microscopy techniques, see FIG. 6 allow for high resolution capture of the entire area of interest with no moving parts. Lensless microscopy is accomplished using blind ptychographic or other methods, such as AI, that allow for computational reconstruction of images from raw sensor data. A light source 600 is present at a sufficient distance from the sequencing assembly (sequencing element, cellular wells, and cellular trap) 610. The light source is created from a variety of options, from an array of pin hole LEDs or lasers, to an optical phased array assembly 605. An optical phased array assembly with on chip lasers 625 would allow not only for apparent x,y translation, but this approach can also allow for complex optical triggering of cells using different wavelength and beam steering. Light then passes through a diffuser 615 before being captured by a complementary metal-oxide semiconductor (CMOS) or other optical sensor 620. In some embodiments, a tiled spectral filter is used for hyper spectral imaging (compatible with LSPR). Fluorescent imaging is done with filtering layer or as tiles on a filter.

Wells and traps may have multiple other features depending on use cases FIG. 4 400. In some cases, magnetically or electrically controlled valves or pumps are added 460. These pumps and/or valves are used to pump reagents into individual wells and not into others. By being individually addressable, specific reagents are added to individual wells as needed. In some cases, a needle 430 is added to inject reagents into the cytoplasm of cells. In some embodiments, electromagnets are added to both the top of the wells or on bottom of the traps as needed 410 470. In a similar fashion, electrodes are added with desired spacing 420. These electrodes are used to detect or send changes in voltage, such as with neurons. In some aspects, cells are allowed to divide in wells and selected through a hole in the sequencing element 480 potentially with an electronically controlled cover. Additionally, in some use cases, individual magnetohydrodynamic pumps can created by applying an magnetic field and placing electrode to have the Lorentz force drive fluid in or out.

In some use cases the valves can be made using magnetic particles larger than the hole size 480 when individually addressable electromagnets are used. For example, electromagnets for cells of interest are turned on and magnetic particles are attracted to areas around the hole covering it and blocking access of other substances through diffusion. Next, the first substance of interest is added. Wells without magnets activated will have the substance diffuse into them. This process can be repeated allowing for large combinations of substances. In some use cases, an external magnetic field can be applied between adding of different substances. The external magnetic field is used to remove magnetic particles that may by chance block wells that should be open. A substance is then added followed by additional magnetic nanoparticles and repeated.

In cases where individually or regionally addressable components are used such as valves, pumps, electromagnets, electrodes, etc., electrical traces following a row and column pattern are used. This shared trace approach allows individual components to be controlled through connections to an external printed circuit board (PCB) using flexible connectors and cables or pins. The PCB will contain best practice components such as driver chips similar to LCD displays controlled by a microcontroller that will have a connection to a computer using USB or other methods.

Alignment of different components is critical for the functioning of the device. Cellular traps are in the order of 10 microns, sequencing probes in the order of 20 microns, and wells in the order 25 microns (although some embodiments may have larger wells). Cellular traps are attached to rectangular blocks of semi-rigid material along the sides and on the bottom in such a way as not to occlude wells unless the method of imaging allows the bottom to be occluded. Similarly, the wells, physically wider and longer than the other components, are also attached to semi-rigid blocks on the top. The sequencing element follows with semi-rigid blocks attached on the top. Using a flexture mechanism or other high precision device, each element is aligned visually using microscopy. If parts tolerances fall outside requirements, physical spacers are placed in between elements and additional features on elements such as a configuration of pegs and holes or grooves and teeth are added that can allow for incremental translation and rotation. Outside the area of valid alignments the teeth or pegs will physically prevent components from coming into contact. If the teeth or pegs are taller than the cells, the components are moved for alignments without displacing or damaging cells and can only lower with complementary grooves or holes. After proper alignment is detected, the spacers are removed and the elements will then be connected.

Another option for alignment is to use hydrophobic dots or patterns in shallow etched wells for a smooth surface places on non-functional areas of components. This allows for parts to align in a similar way. Using a spacer or potentially magnetic levitation, components are aligned with microscopy and spacers removed. The interaction of hydrophobic and hydrophilic areas keeps the parts aligned and the size and spacing of the dots or patterns allows micron level alignment.

If all parts have tight tolerances, in the order of a few microns depending on use case, parts can contain raised markings paired with complementary lowered markings on other parts. In this way the sequencing element is precisely aligned to wells by rough manual alignment. Similarly, the cellular traps are precisely aligned to wells. If the height of the markings is higher than cells, the wells are added and then moved and dropped into place.

Depending on exact implementation, the sequencing element could be aligned perfectly with each well, or it could be imperfectly aligned (some probes may be outside of wells), or aligned with overlapping wells. Cellular wells are unlikely to be fully populated, so using the absences in observed locations, imperfectly aligned wells are aligned using pattern matching algorithms. In the case of overlapping wells, the sequencing data will have multiple barcodes per cell. These multiple barcodes are used to filter out cells into individual wells through probability based models such as maximum likelihood estimation.

Depending on configuration and use, reagents for reverse transcription polymerase chain reaction (RT-PCR) are pre-loaded into cellular wells, in cellular trap media, or through pumps. Reagents may include, but are not limited to, reverse transcriptase, DNA ligase, DNA polymerase, nucleosides, and PCR primers. After experiments and observations are complete and components are secure, meaning there will be minimal or no leakage of mRNA or DNA from one well to another when exposed to repeated temperature changes, cells are lysed using methods such as, but not limited to, chemical lysis (added through pumps or semipermeable membranes), magnetic lysis (alternating magnetic fields with magnetic cells for example), or thermal lysis. Next, using best practices for the probe design and the target of interest, PCR/RT-PCR is performed to amplify the target or targets of interest. The solution in the wells is then collected by disassembling the device and collecting the fluid directly or after a wash of an appropriate solvent and then is collected. In some embodiments, addressable pumps can remove the solution that can then be collected. The cDNA library solution is then prepared using best practices and is then sequenced.

Although the assembly can be adapted to a traditional bench-top thermal cycler, the footprint of the device is greatly reduced by using the same approach as used in creation of single cell traps, as shown in FIG. 5. A 3D printed or otherwise machined part 555 allows for the insertion of the single cell trap 592, attachment of cellular wells 591, and sequencing element 590 allowing for assembly and disassembly with clamping or other methods 585. The part may contain multiple ports for injection of reagents 570, CO2 or other gasses for cellular incubation 560, flow of cells (pumping back and forth from a larger reservoir) 565, washing off excess cells 565, adding reagents 570, and for collecting solution via pipette 580. For incubation and loading of cellular traps, a temporary lid may be used to keep appropriate atmospheric conditions. The part is also a water block that is connected to a similar or same device as mentioned previously to precisely control temperature 575. In some aspects, this device is used for incubation of cells, lysis of cells, and thermal cycling for PCR reactions. Additionally, the base of the part may contain lighting such as diffuse LEDs, electroluminescence, laser diffusion through an angles port, or components for lensless microscopy as previously mentioned.

After the cDNA library is sequenced, barcodes are recovered and corrected for errors using standard bioinformatics tools such as UMI-tools. These barcodes, attached to every valid transcript, have a one-to-one mapping with the cellular observations made in cellular wells allowing single cells to be sequenced with paired cellular observations. This novel single-cell sequencing method allows for many potential applications from high throughput affinity and binding databases of antibodies or TCRs to transcriptomics of cells under different chemical gradients to transcriptomic differences neurons exposed to different signals from electrodes to studying the affects of different combinations of therapeutic drugs in multiple types of cells at once.

An illustrative device is described below. In one embodiment, the device is described as a single-cell observation and barcoding complex. The complex comprises a platform defining one or more cellular wells, each cellular well capable of capturing a single cell, and wherein each cellular well aligns to one species of a barcoded solid phase PCR probe. The barcoded probe is a member of a sequencing element, as previously described, of multiple solid-phase barcoded solid phase PCR probes. When single cells of interest are applied to the complex, they can be captured in the target cellular well and observed. The single cells can be lysed in their respective cellular well, and steps to create barcoded cDNA sequences taken (as described above). As such, the barcoded cDNA sequences are collected and cDNA sequences mapped to cellular observations, as described herein, related to each individual cellular well. In some aspects the single-cell observation and barcoding complex platform is formed from thermally stable glass, although any material compatible with an aqueous environment and temperatures up to approximately 100° C. can be used. Platform materials should also correspond with the type of cellular observation planned for the device, for example, where observations require visible light, the material is typically transparent.

Besides the previously mentioned use cases, these methods described herein can also be used to create personalized treatments for cancer, autoimmune diseases, and allergies. These methods can also be used to create fully humanized antivenoms.

In the case of cancer, cellular traps are made to capture cancer cells or in some cases larger pieces of tissue such as micrometastases. Cancer is often highly heterogeneous and individual cells transcriptomes are sequenced with the sequence element designed to sequence all mRNA for use in identifying candidate drugs or creating personalized vaccines such as with mRNA technology. As combination drugs are more effective, because it is less likely for cancer to evolve around all drugs simultaneously, addressable pumps are used to deliver combinations of drugs to individual cancer cells or groups of cancer cells to find combinations that are effective in killing all cells. Trapped cancer cells or groups of captured cells are used to study the tumor microenvironments. Immune cells are flowed over the traps and observed for interactions. Interacting immune cells are captured with wells placed over them and trapped cancer cells where observations are made. If the immune cells are magnetically labeled, they are pulled to the top of the well and the cancer cells removed. In this way, transcriptomes, B-cell receptors (BCRs), or T-cell receptors (TCRs), are sequenced with observations. After common targets are identified through bioinformatics and AI, custom cellular traps of cancer proteins (likely surface proteins) or pMHC (matched to patient specific alleles and cancer specific epitopes both class I and class II) are created. B-cells are flowed across the protein traps and T-cells over the pMHC traps (may be a separate or the same trap array). Camelid VHH B-cells (nanobody producing B-cells) can also follow the same process and would be useful in creating patient specific CAR (chimeric antigen receptor) cells. Cells are allowed to divide for a few rounds and then clones are selected. Sequencing then happens as previously mentioned. Cloned cells can then be passed over modified traps, illustrated in FIG. 7 to check for potentially deadly cross-reactivity. The modified traps (proteins, organoids, etc.) 705 are created to line up to a microfluidic channel 700 (separate piece that is aligned with traps), using laser etching or other methods, where a single cell 710 must interact with each trap and are pushed from one trap to another with pumped fluid. The goal of the serial path of traps is to account for as much as the proteome or patient specific pMHC as possible. If a clone cell makes it through the channel without observed interactions, the chance of cross-reactivity is greatly reduced. Multiple cells are passed at once if trapped cells are removed using an additional microfluidic channel similar to the previous well design 725. A cell 715 bound to a trap 720 are removed through addressable electromagnets as before 730. Additionally, valves in both the upper 735 and the lower 740 channel are used to control flow to flush cells into the upper chamber 745. Potentially multiple lanes are on one observed part allowing for multiple different cell lines to be passed at once. Effectively, clones are used to create antibody treatments, CAR T-cells, CAR NK cells, or find CD4+ and CD8+ combinations (potentially modified to ignore cancer immunosuppression) that directly target cancer cells. These found or created immune cells can then be tested with cancer cells in cellular traps as mentioned earlier for effectiveness. Regulatory cells can often be protective of cancer cells. Using a method described in the next paragraph, these cells are directly targeted for removal.

In the case of autoimmune diseases, MHC and allelic matched disease affected cells of interest (ideally clones from the patient) are added to cellular traps that bind to the cells of interest. In some cases, multiple cells or multiple types of cells are needed, and in this case cellular traps are made close together potentially, with different proteins or other targets of interest to capture different combinations of cells. In some aspects, larger structures like organoids are captured by cellular traps. Isolated effector cells such as B-cells or T-cells and immunomodulating compounds such as certain cytokines (to increase the likelihood of killing trapped cells) are added and flowed over the traps. Interacting effector cells are at least temporarily bound, for example a cytotoxic T-cell takes around 10 minutes to deliver an apoptosis signal to a cell with up to 4 hours for the targeted cell to undergo apoptosis. In some cases, it is appropriate to allow T-cells to migrate around the cellular traps triggering apoptosis on multiple cells, in this case using image tracking methods and/or AI, individual T-cells are tracked before wells are aligned for sequencing. After observations, cells are sequenced and mapped BCRs or TCRs, from either B-cells or T-cells respectively, that killed trapped cells are recombinantly produced (ideally in just variable regions). These BCRs/antibodies or TCRs are used to create new cellular traps (likely with multiple targets of interests). On the new cellular traps B-cells are flowed over the traps, captured, and affinity measurements are collected as before. These B-cells are first selected from magnetic beads containing poly BCRs/antibodies or poly TCRs. In this way B-cells that bind to any constant region and non-specific variable regions are removed. In some aspects, cells are allowed to divide a few rounds and candidate clones are selected and picked for clonal expansion. Trap cells are then sequenced as before. In some aspects, AI methods are used to identify what sequences will best bind to the idiotype with minimal cross-reactivity as wells as removing weak binding with magnetic fields as previously mentioned. Clonal B-cell candidates can then flow over a new cellular trap of the human proteome to minimize cross-reactivity (potentially the serial configuration mentioned previously). Once confirmed, the BCR/antibody are used to create monoclonal antibodies, or CAR T-cells to target disease causing immune cells. As mentioned before with cancer, a similar approach could be used with pMHC to find T-cells/TCRs to delete disease causing cells.

In the case of allergies, cellular traps are constructed with known allergens of interest for a patient. For example, peanuts contain 12 known allergens with multiple isoforms. These allergens are printed on a microarray. As T-cells play a role in allergens as well, pMHC (class I and class II matching patient alleles) traps are made as using AI or other methods to identify allergen peptides. Using the laser ablation technique previously mentioned cellular traps specific for allergy causing adaptive immune cells are made. Isolated immune cells of interest from a patient, such as B-cells that produce IgE, are passed over the array and allergen reactive cells are trapped. The wells are aligned and individual immune cells of interest are isolated and sequenced isolating BCRs (in some cases a pMHC approach as mentioned before and TCR sequencing is performed). The sequenced BRCs/antibodies are added to a new protein microarray and cellular traps created using methods previously described. Filtered B-cells (or T-cells for pMHC) using the previously described method are passed over the patient specific antibody traps of interests and B-cells are captured. B-cells are directly captured from traps or allowed to divide a few times in larger wells and colonies of interest are selected, for example, using affinity calculations, for clonal expansion. These clones can then flow over a new cellular trap with the antibody of interest as well as the proteome to avoid cross-reactivity. The clones and/or sequences are used to create monoclonal antibodies or CAR T-cells to target allergen reactive cells.

Embodiments herein include building large scale binding affinity databases with arbitrary diverse protein microarrays such as human or mice proteomes. The same approach herein is used in the creation of fully humanized antivenoms. Venoms, whether they are neurotoxic, hemotoxic, cytotoxic, etc. are a complex mixer of multiple different compounds with the vast majority being proteins or peptides. Peptide/protein microarrays as illustrated in FIG. 8 is created from venom 805 collected from one or more members of a species or subspecies of interest (for example, for use with venoms delivered by venomous spiders, scorpions, bees, wasps, centipedes, jellyfish, stonefish, stingrays, snakes, lizards, frogs, and the like) 800. Using best practices for fractionation techniques 810, such as high-performance liquid chromatography (HPLC), are used to isolate and collect individual components of a venom. These individual components can then be used to print 815 a venom specific peptide/protein microarray 820 using best practices. After the microarray is constructed, B-cells are flowed over the microarray using methods previously described allowing for the collection of binding, affinity, and sequences of specific antibodies. Similar checks for cross-reactivity minimization and affinity maturation is performed as previously mentioned. The combination of discovered antibodies are combined to make a safe an effective antivenom without the need for potentially dangerous traditional antivenoms.

Unlike previous methods and inventions, high throughput single-cell sequencing with cellular observations is possible with this invention allowing for many useful applications. Cellular traps allow precise alignment of cells of interest that can then be sequenced. This invention allows large scale collection of binding and affinity data from different cells, such as B-cell or T-cells, that are paired with sequences allowing for large training sets for AI models. A high throughput way of observing interactions of cancer cells with immune cells. This invention also allows for the identification of disease-causing cells in autoimmune diseases and allergies. The selection of B-cells and T-cells allows for personalized biologics for use in cancer, autoimmune diseases, and allergies as well as fully humanized antivenoms.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A single-cell observation and sequencing system comprising:
a platform defining one or more cellular wells, each cellular well configured to capture a single cell of a target of interest;
a sequencing element comprising one or more barcoded solid phase polymerase chain reaction (PCR) probes;
wherein each cellular well is configured to align with a unique species of one of said barcoded solid phase PCR probes; and
wherein each cellular well is further configured to allow for cellular observation, cellular lysing, and genetic sequencing therein to provide barcoded complementary DNA (cDNA) sequences with mapped cellular observations for each cell within said one or more cellular wells.

2. The system of claim 1, further comprising:
one or more cellular traps; and
wherein each cellular trap is aligned with one of said one or more cellular wells and is configured to bind to said single cell of a target of interest.

3. The system of claim 2, wherein:
each cellular well comprises a center hole; and
each cellular trap comprises a microfluidic valve configured to open and close over a respective center hole via a microfluidic pump.

4. The system of claim 1, wherein:
said sequencing element further comprises:
a molecular spacer;
a shared primer sequence;
a molecular barcode; and
an adapter sequence.

5. The system of claim 4, wherein:
said sequencing element further comprises first and second potential cleavage sites positioned downstream of said molecular spacer and upstream of said shared primer sequence.

6. The system of claim 5, wherein:
each said solid phase PCR probe further comprises a run ID segment; and
said first potential cleavage site comprises a cleavage site on said run ID segment allowing for reusability of said solid phase PCR probe.

7. The system of claim 4, wherein:
said sequencing element further comprises a unique molecular identifier (UMI) site positioned downstream of said shared primer sequence and upstream of said adapter sequence.

8. The system of claim 2, wherein:
each cellular trap is configured so as to allow for testing said single cell of said target of interest for affinity data using one or more magnetic field.

9. The system of claim 2, wherein:
each cellular trap further comprises a light source configured so as to allow for testing said single cell of said target of interest for affinity data with plasmin resonance; and
each cellular trap is configured so as to allow for removal of surrounding cells using one or more magnetic field.

10. The system of claim 1, wherein:
the system is scalable such that said cellular observations are mapped to said barcoded cDNA sequences massively in parallel.

11. The system of claim 1, further comprising:
a lensless microscope configured to allow a user to view all of said one or more cellular wells at once.

12. A method of single-cell observation and sequencing comprising:
providing a platform defining one or more cellular wells, each cellular well configured to capture a single cell;
providing a sequencing element comprising a barcoded solid phase PCR probe, wherein each cellular well is configured to align with a unique species of said barcoded solid phase PCR probe;
capturing individual cells of a target of interest within said one or more cellular wells, one single cell of said target of interest per each cellular well;
recording cellular observation data in association with each said single cell of said target of interest within each cellular well;
lysing each said single cell of said target of interest within each cellular well;
sequencing genetic material of said lysed cell within each cellular well, creating barcoded cDNA sequences; and
mapping cellular observations for each cell of said target of interest within said one or more cellular wells to said barcoded cDNA sequences.

13. The method of claim 12, further comprising:
providing one or more cellular traps, wherein each cellular trap is aligned with one of said one or more cellular wells and is configured to bind to said single cell of a target of interest.

14. The method of claim 13, wherein each cellular well comprises a center hole and each cellular trap comprises a microfluidic valve configured to open and close over a respective center hole, the method further comprising:
for each cellular well, engaging a microfluidic pump to open said microfluidic valve and allow said single cell of said target of interest through said cellular trap and into said cellular well.

15. The method of claim 13, further comprising:
applying one or more micro electromagnets to each single cell of said target of interest within said one or more cellular wells; and
calculating binding kinetics.

16. The method of claim 13, further comprising:
applying one or more light source to each single cell of said target of interest within said one or more cellular wells to test for affinity data with plasmin resonance; and
calculating binding kinetics.

17. The method of claim 12, further comprising:
mapping said cellular observations to said barcoded cDNA sequences massively in parallel; and
storing said massively parallel barcoded cDNA sequences and mapped cellular observations to a cellular database.

18. The method of claim 12, wherein said target of interest comprises cancer cells, cells associated with autoimmune diseases, or cells associated with allergies; and said cellular observations are selected from the group consisting of: interactions with drugs, interactions with proteins, interactions with other cells, interactions with biological materials, binding kinetics, affinity, and combinations thereof.

19. The method of claim 12, wherein said target of interest comprises venom from a species or subspecies of interest in development of fully humanized antivenom.

20. The method of claim 12, wherein said method is utilized to minimize cross-reactivity.
